(19) United States
(12) Patent Application Publication
(10) Pub. No.: US 2024/0377629 A1
(43) Pub. Date: Nov. 14, 2024

(54) ENDOSCOPIC IMAGING DEVICE, ENDOSCOPE, AND METHOD FOR MANUFACTURING ENDOSCOPIC IMAGING DEVICE

(71) Applicant: OLYMPUS MEDICAL SYSTEMS CORP., Tokyo (JP)

(72) Inventor: Toshinari MIZUKAMI, Tachikawa-shi (JP)

(73) Assignee: OLYMPUS MEDICAL SYSTEMS CORP., Tokyo (JP)

(21) Appl. No.: 18/659,443

(22) Filed: May 9, 2024

Related U.S. Application Data
(60) Provisional application No. 63/465,948, filed on May 12, 2023.

(51) Int. Cl. G02B 23/24; A61B 1/00; G02B 7/02

(52) U.S. Cl. CPC G02B 23/243; A61B 1/00114; A61B 1/00124; G02B 7/02

(57) ABSTRACT

What is provided is an endoscopic imaging device that can curb an increase in the number of assembly steps. The endoscopic imaging device includes an optical system, a tubular holding frame configured to accommodate a part of the optical system therein and to extend in an optical axis direction, an electronic component having a wiring portion and held in the holding frame, and a substrate having a winding portion configured to extend in a circumferential direction along an outer circumferential surface of the holding frame, and a straight portion configured to extend from the winding portion to one side in the optical axis direction, wherein the substrate has a conductive portion disposed across the winding portion and the straight portion, and the wiring portion is connected to the conductive portion disposed in the winding portion.

### BACKGROUND OF THE INVENTION

### Field of the Invention

The present invention relates to an endoscopic imaging device, an endoscope, and a method for manufacturing an endoscopic imaging device.

### Description of Related Art

Conventionally, in a holding frame that accommodates an optical system therein, an endoscopic imaging device that changes a focal point of a subject by moving a movable frame that holds an optical system back and forth in an optical axis direction using an electromagnetic actuator is known (for example, Patent Document 1).

### PATENT DOCUMENTS

[Patent Document 1] Japanese Unexamined Patent Application, First Publication No. 2017-63845

### SUMMARY OF THE INVENTION

In the above-described endoscopic imaging device, a configuration in which a wiring portion pulled out of the electromagnetic actuator disposed on the distal end side of the endoscopic imaging device is electrically connected to a cable disposed on the proximal end side of the endoscopic imaging device via a substrate or the like fixed to a lens barrel part may be adopted. In an assembly process of the endoscopic imaging device having such a configuration, when the optical system is adjusted by rotating the holding frame with respect to the lens barrel part, since a circumferential position of a portion of the wiring portion pulled out of the electromagnetic actuator with respect to a circumferential position of the substrate varies according to the endoscopic imaging device, it was necessary to adjust a length of the wiring portion and to remove an insulation coating from the wiring portion. Therefore, work of connecting the wiring portion and the substrate becomes complicated, and there is a possibility that the number of assembly steps for the endoscopic imaging device will increase.

In view of the above circumstances, one of the objects of the present invention is to provide an endoscopic imaging device, an endoscope, and a method for manufacturing an endoscopic imaging device in which any increase in the number of assembly steps is curbed.

In order to achieve the above object, an endoscopic imaging device according to one aspect of the present invention includes an optical system configured to form an optical image, a tubular holding frame configured to accommodate a part of the optical system therein and to extend in an optical axis direction, an electronic component having a wiring portion and held in the holding frame, and a substrate having a winding portion configured to extend in a circumferential direction along an outer circumferential surface of the holding frame, and a straight portion configured to extend from the winding portion to one side in the optical axis direction, wherein the substrate has a conductive portion disposed across the winding portion and the straight portion, and the wiring portion is connected to the conductive portion disposed in the winding portion.

An endoscope according to one aspect of the present invention includes an inserting part inserted into a subject, and an endoscopic imaging device provided at a distal end of the inserting part, wherein the endoscopic imaging device includes an optical system which forms an optical image, an electronic component having a wiring portion, a tubular holding frame which accommodates a part of the optical system therein and extends in an optical axis direction, and a substrate having a winding portion which extends in a circumferential direction along an outer circumferential surface of the holding frame, and a straight portion which extends from the winding portion to one side in the optical axis direction, and the wiring portion is connected to a portion of the winding portion near a position at which the wiring portion is pulled out of the electronic component.

A method for manufacturing an endoscopic imaging device according to one aspect of the present invention is a method for manufacturing an endoscopic imaging device which includes an optical system that forms an optical image, a tubular holding frame that accommodates a part of the optical system therein and extends in an optical axis direction, an electronic component having a wiring portion and held in the holding frame, a lens barrel part that holds the holding frame, and a substrate having a winding portion that extends in a circumferential direction along an outer circumferential surface of the holding frame, and a straight portion that extends from the winding portion to one side in the optical axis direction, wherein the wiring portion is connected to a portion of the winding portion near a position at which the wiring portion is pulled out of the electronic component, the method including a first step of adjusting a position of the optical system by rotating the holding frame in the circumferential direction with respect to the lens barrel part, a second step of fixing the holding frame to the lens barrel part, a third step of fixing the straight portion to the lens barrel part and fixing the winding portion to the holding frame, and a fourth step of connecting the wiring portion to the winding portion.

According to the present invention, it is possible to provide an endoscopic imaging device, an endoscope, and a method for manufacturing an endoscopic imaging device that can curb an increase in the number of assembly steps.

### BRIEF DESCRIPTION OF THE DRAWINGS

FIG. 1 is an external view showing an endoscope equipped with an endoscopic imaging device according to an embodiment.

FIG. 2 is a perspective view showing the endoscopic imaging device according to the embodiment.

FIG. 3 is a cross-sectional view showing the endoscopic imaging device according to the embodiment.

FIG. 4 is a side view showing a part of a winding portion of the embodiment.

FIG. 5 is a cross-sectional view showing the winding portion of the embodiment, and is a cross-sectional view taken along line V-V in FIG. 4.

FIG. 6 is a flowchart showing a method for manufacturing an endoscopic imaging device according to an embodiment.

FIG. 7 is a first perspective view showing an assembly process of the endoscopic imaging device according to the embodiment.

FIG. 8 is a second perspective view showing an assembly process of the endoscopic imaging device according to the embodiment.

FIG. 9 is a side view showing a part of a winding portion of a first modified example of the embodiment.

FIG. 10 is a cross-sectional view showing the winding portion of the first modified example of the embodiment, and is a cross-sectional view taken along line X-X in FIG. 9.

FIG. 11 is a side view showing a part of a winding portion of a second modified example of the embodiment.

FIG. 12 is a side view showing a part of a winding portion of a third modified example of the embodiment.

### DETAILED DESCRIPTION OF THE INVENTION

Hereinafter, an endoscopic imaging device, an endoscope, and a method for manufacturing an endoscopic imaging device according to embodiments of the present invention will be described with reference to the drawings. The scope of the present invention is not limited to the following embodiments, and can be arbitrarily modified within the scope of the technical idea of the present invention. Further, in the following drawings, in order to make each configuration easier to understand, the scales, number, and the like of respective structures may be different from those of actual structures.

An endoscope 1 is a device inserted into a patient's lumen to observe and treat an affected area. In the following description, in the endoscope 1, the side inserted into the patient's lumen is referred to as the “distal end side (distal side),” and the side opposite to the side inserted into the patient's lumen is referred to as the “proximal end side (proximal side).”

In each of drawings, a Z axis is indicated as appropriate. The Z-axis indicates a direction in which an optical axis J of the embodiment described below extends. The optical axis J shown in each of drawings is a virtual axis. In the following description, the direction in which the optical axis J extends, that is, the direction parallel to the Z axis, will be referred to as an “optical axis direction.” A radial direction centered on the optical axis J is simply referred to as a “radial direction.” A circumferential direction centered on the optical axis J is simply referred to as a “circumferential direction.” The side (the +Z side) in the optical axis direction toward which an arrow of the Z-axis faces is the “distal end side (the distal side),” and the side (the −Z side) in the optical axis direction opposite to the side toward which the arrow of the Z-axis faces is the “proximal end side (the proximal side).” In the following description, the side (the +Z side) in the optical axis direction toward which the arrow of the Z-axis faces will be referred to as the “distal end side” or “the other side in the optical axis direction.” The side (the −Z side) in the optical axis direction opposite to the side toward which the arrow of the Z-axis faces is referred to as the “proximal end side” or “one side in the optical axis direction.”

The circumferential direction is indicated by an arrow θ in each of the drawings. The side (the +θ side) in the circumferential direction toward which the arrow θ faces is referred to as “one side in the circumferential direction.” The side (the −θ side) in the circumferential direction opposite to the side toward which the arrow θ faces is referred to as “the other side in the circumferential direction.” One side in the circumferential direction is the side that moves clockwise around the optical axis J when seen from the distal end side. The other side in the circumferential direction is the side that moves counterclockwise around the optical axis J when seen from the distal end side.

### First Embodiment

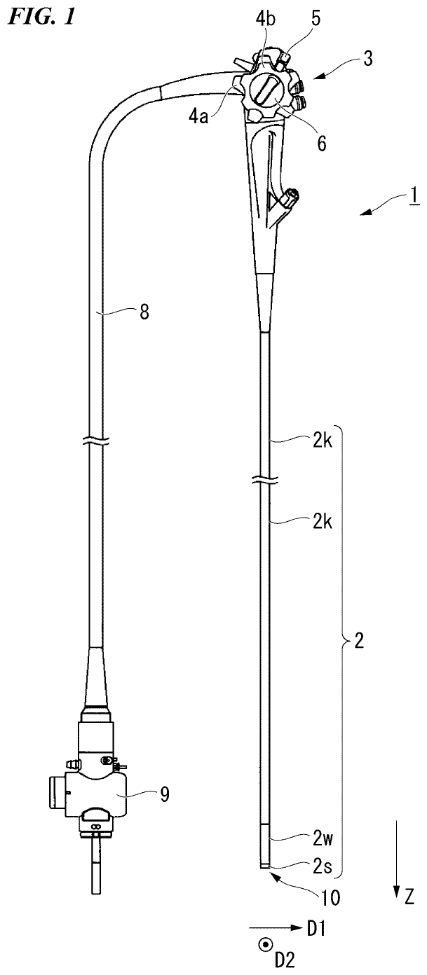

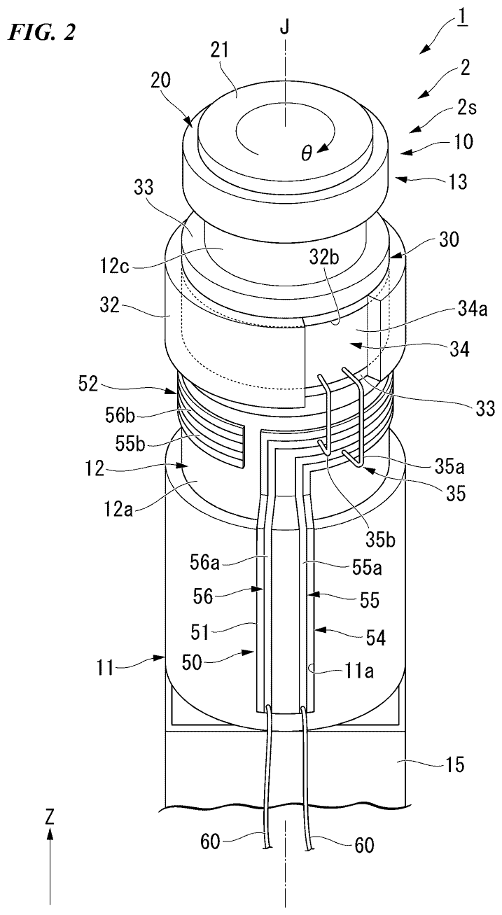

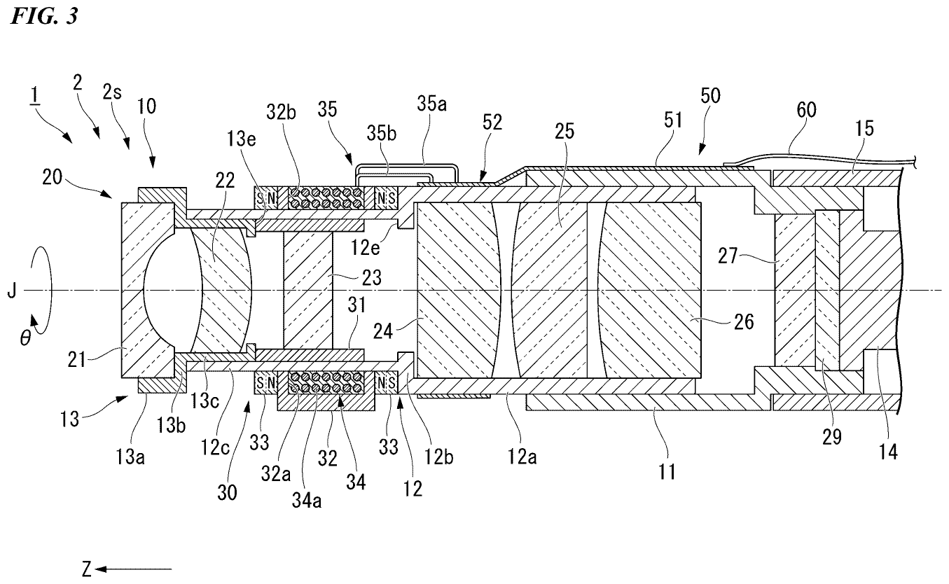

FIG. 1 is an external view showing an endoscope 1 equipped with an endoscopic imaging device 10 of this embodiment. FIG. 2 is a perspective view showing the endoscopic imaging device 10 of this embodiment. FIG. 3 is a cross-sectional view showing the endoscopic imaging device 10 of this embodiment.

As shown in FIG. 1, the endoscope 1 includes an inserting part 2 inserted into a subject, an operation part 3 coupled to the proximal end side of the inserting part 2, a universal cord 8 that extends from the operation part 3, a connector 9 connected to the universal cord 8, and the endoscopic imaging device 10. The endoscope 1 is electrically connected to an external device such as a control device and a lighting device (not shown) via the connector 9.

The inserting part 2 includes, in order from the distal end side, a distal end portion 2s, a curved portion 2w, and a flexible tube portion 2k. The inserting part 2 is formed to be elongated in the optical axis direction. The endoscopic imaging device 10 is provided at the distal end of the distal end portion 2s. That is, the endoscopic imaging device 10 is provided at the distal end of the inserting part 2. The curved portion 2w can be curved in each of a first direction D1 and a second direction D2 that are orthogonal to the optical axis direction. The flexible tube portion 2k connects the curved portion 2w and the operation part 3. The flexible tube portion 2k has a tubular shape that extends in the optical axis direction. A cable 60 shown in FIG. 2 that electrically connects the connector 9 and the endoscopic imaging device 10 is accommodated inside the flexible tube portion 2k.

As shown in FIG. 1, the operation part 3 includes curving operation knobs 4a and 4b that curve the curved portion 2w of the inserting part 2, a fixing lever 5 that fixes a rotational position of the curving operation knob 4a, and a fixing knob 6 that fixes a rotational position of the curving operation knob 4b. By rotating the curving operation knobs 4a and 4b, the curved portion 2w curves in the first direction D1 and the second direction D2. Thus, an observation direction of the endoscopic imaging device 10 can be changed, and insertability of the distal end portion 2s into the subject can be improved. As will be described below, a movable frame 31 can be moved in the optical axis direction and a focal point of the subject can be changed by operating a button (not shown) of the operation part 3 or an external device (not shown).

As shown in FIGS. 2 and 3, the endoscopic imaging device 10 includes a lens barrel part 11, a holding frame 12, an objective lens holding part 13, a reinforcing frame 15, an electronic device 14, an optical system 20, an electronic component 30, and a substrate 50.

As shown in FIG. 2, the lens barrel part 11 has a tubular shape that extends in the optical axis direction. In this embodiment, a portion of the lens barrel part 11 that is closer to the distal end side than the reinforcing frame 15 has a substantially cylindrical shape that extends in the optical axis direction with the optical axis J as the center. A portion of the lens barrel part 11 located inside the reinforcing frame 15 has a square tube shape that extends in the optical axis direction. As shown in FIG. 3, the lens barrel part 11 is open on both sides in the optical axis direction. As shown in FIG. 2, a substrate mounting portion 11a is provided on the lens barrel part 11. The substrate mounting portion 11a is a recess that is recessed radially inward from an outer circumferential surface of the lens barrel part 11. The substrate mounting portion 11a extends in the optical axis direction.

As shown in FIG. 3, the holding frame 12 has a tubular shape that extends in the optical axis direction. In this embodiment, the holding frame 12 has a substantially cylindrical shape that extends in the optical axis direction with the optical axis J as the center. The holding frame 12 may have a rectangular tube shape that extends in the optical axis direction. The holding frame 12 is open on both sides in the optical axis direction. The holding frame 12 accommodates a part of the optical system 20 therein. The holding frame 12 includes a lens accommodation portion 12a, a first annular portion 12b, and a movable frame accommodation portion 12c.

The lens accommodation portion 12a has a substantially cylindrical shape that extends in the optical axis direction with the optical axis J as the center. A proximal end portion of the lens accommodation portion 12a is accommodated inside the lens barrel part 11. In this embodiment, the lens accommodation portion 12a is fixed to the lens barrel part 11 with an adhesive (not shown). Thus, the lens barrel part 11 holds the holding frame 12.

The first annular portion 12b has an annular shape centered on the optical axis J. A radially outer edge portion of the first annular portion 12b is connected to the distal end of the lens accommodation portion 12a in the optical axis direction. A dimension of an outer diameter of the first annular portion 12b is the same as that of an outer diameter of the lens accommodation portion 12a. An inner diameter of the first annular portion 12b is smaller than an inner diameter of the lens accommodation portion 12a.

The movable frame accommodation portion 12c has a substantially cylindrical shape that extends in the optical axis direction with the optical axis J as the center. The movable frame accommodation portion 12c extends from the first annular portion 12b to the distal end side. An outer diameter of the movable frame accommodation portion 12c is smaller than the outer diameter of the first annular portion 12b. An inner diameter of the movable frame accommodation portion 12c is larger than the inner diameter of the first annular portion 12b. In a surface of the first annular portion 12b that faces the distal end side, a portion that is located radially inner than the movable frame accommodation portion 12c is a first restriction surface 12e. When seen in the optical axis direction, the first restriction surface 12e has an annular shape centered on the optical axis J.

The objective lens holding part 13 has a substantially cylindrical shape that extends in the optical axis direction with the optical axis J as the center. The objective lens holding part 13 is open on both sides in the optical axis direction. The objective lens holding part 13 is fixed to the distal end portion of the holding frame 12. The objective lens holding part 13 has a first tubular portion 13a, a second annular portion 13b, and a second tubular portion 13c.

The first tubular portion 13a has a substantially cylindrical shape that extends in the optical axis direction with the optical axis J as the center. The first tubular portion 13a is disposed closer to the distal end side than the holding frame 12. An outer diameter of the first tubular portion 13a is larger than the outer diameter of the movable frame accommodation portion 12c.

The second annular portion 13b has an annular shape centered on the optical axis J. A radially outer edge portion of the second annular portion 13b is connected to an end portion of the first tubular portion 13a on the proximal end side in the optical axis direction. An inner diameter of the second annular portion 13b is smaller than the inner diameter of the movable frame accommodation portion 12c. A surface of the second annular portion 13b that faces the proximal end side is in contact with the distal end of the movable frame accommodation portion 12c in the optical axis direction.

The second tubular portion 13c has a substantially cylindrical shape that extends in the optical axis direction with the optical axis J as the center. The second tubular portion 13c extends from the radially inner edge of the second annular portion 13b to the proximal end side. The second tubular portion 13c is disposed inside a portion of the movable frame accommodation portion 12c on the distal end side. A surface of the second tubular portion 13c that faces the proximal end side is a second restriction surface 13e. The second restriction surface 13e has an annular shape centered on the optical axis J when seen in the optical axis direction. The second restriction surface 13e faces the first restriction surface 12e in the optical axis direction.

The reinforcing frame 15 has a square tube shape that extends in the optical axis direction. In this embodiment, the reinforcing frame 15 has a rectangular tube shape. The reinforcing frame 15 surrounds the optical axis J from the outside in the radial direction. When seen in the optical axis direction, an inner circumferential surface of the reinforcing frame 15 has a rectangular shape centered on the optical axis J. The reinforcing frame 15 is fixed to an outer circumferential surface of a rectangular tube portion of the lens barrel part 11 on the proximal end side. The reinforcing frame 15 surrounds the electronic device 14 from the outside in the radial direction. The reinforcing frame 15 can protect and reinforce the electronic device 14. When seen in the optical axis direction, the inner circumferential surface of the reinforcing frame 15 may have an arc shape centered on the optical axis J.

The electronic device 14 is a substrate on which electronic components such as capacitors are mounted. The electronic device 14 may be a laminated substrate. The electronic device 14 is fixed to the proximal end side of the lens barrel part 11. An imaging element 29 is mounted on the electronic device 14 and held in the lens barrel part 11. The imaging element 29 is an image sensor such as a CCD or a CMOS. The imaging element 29 converts an optical image of the subject formed by the optical system 20 into a video signal. The imaging element 29 is connected to an external device (not shown) via a cable (not shown) and the connector 9.

The optical system 20 forms an optical image of the subject. In this embodiment, the optical system 20 is configured of seven lenses. The optical system 20 includes an objective lens 21, a first lens 22, a movable lens 23, a second lens 24, a third lens 25, a fourth lens 26, and a fifth lens 27. The lenses are disposed in the following order of the objective lens 21, the first lens 22, the movable lens 23, the second lens 24, the third lens 25, the fourth lens 26, and the fifth lens 27 from the distal end side to the proximal end side. The configuration of the optical system 20 is not limited to the configuration of this embodiment, and for example, the number of lenses constituting the optical system 20 may be 6 or less, or 8 or more.

The objective lens 21 is held on an inner circumferential surface of the first tubular portion 13a. A surface of the objective lens 21 that faces to the distal end side is exposed to the outside of the endoscopic imaging device 10. The objective lens 21 may be configured of a plurality of lens groups. The first lens 22 is accommodated inside the second tubular portion 13c. The first lens 22 is held on the inner circumferential surface of the second tubular portion 13c.

The movable lens 23 is accommodated inside the movable frame accommodation portion 12c. The movable lens 23 is held on the inner circumferential surface of the movable frame 31 which will be described below. In the optical axis direction, the movable lens 23 is disposed between the first restriction surface 12e and the second restriction surface 13e. The movable lens 23 may be configured of a plurality of lens groups.

Each of the second lens 24, the third lens 25, and the fourth lens 26 is held on the inner circumferential surface of the lens accommodation portion 12a. The fifth lens 27 is held at a portion of the inner circumferential surface of the lens barrel part 11 on the proximal end side. The optical image of the subject formed by the optical system 20 is converted into a video signal by the imaging element 29 and transmitted to an external device (not shown) via a cable (not shown) and the connector 9.

The electronic component 30 is held by the holding frame 12. More specifically, the electronic component 30 is held in the movable frame accommodation portion 12c. The electronic component 30 is electrically connected to the substrate 50. A current is supplied to the electronic component 30 from an external device (not shown) via the substrate 50 and the cable 60. In this embodiment, the electronic component 30 is an electromagnetic actuator that moves the movable lens 23 in the optical axis direction using a magnetic force. In this embodiment, the focal point of the subject in the endoscope 1 can be changed by moving the movable lens 23 in the optical axis direction. The configuration of the electronic component 30 is not limited to this embodiment, and for example, the electronic component 30 may be a heater or the like that curbs dew condensation in the optical system 20. The electronic component 30 includes the movable frame 31, a yoke 32, a pair of magnets 33, and a coil part 34.

The movable frame 31 has a substantially cylindrical shape that extends in the optical axis direction with the optical axis J as the center. The movable frame 31 is open on both sides in the optical axis direction. The movable frame 31 is configured of a magnetic body. The movable frame 31 is disposed inside the movable frame accommodation portion 12c. That is, the movable frame 31 is disposed inside the holding frame 12. A part of the outer circumferential surface of the movable frame 31 is in contact with the inner circumferential surface of the movable frame accommodation portion 12c. The movable frame 31 is movable in the optical axis direction. In the optical axis direction, the movable frame 31 is disposed between the first restriction surface 12e and the second restriction surface 13e. When seen in the optical axis direction, the movable frame 31 overlaps the first restriction surface 12e and the second restriction surface 13e. A dimension of the movable frame 31 in the optical axis direction is smaller than a dimension in the optical axis direction between the first restriction surface 12e and the second restriction surface 13e. Thus, the movable frame 31 is movable in the optical axis direction between the first restriction surface 12e and the second restriction surface 13e. The movable lens 23 is held on the inner circumferential surface of the movable frame 31. That is, the movable frame 31 holds a part of the optical system 20 therein. In this embodiment, the movable lens 23 can be moved in the optical axis direction and the focal point of the subject in the endoscope 1 can be changed by moving the movable frame 31 in the optical axis direction.

The yoke 32 has a substantially annular shape centered on the optical axis J. In this embodiment, the yoke 32 is configured of a magnetic body. The yoke 32 is provided outside the movable frame 31. The yoke 32 surrounds the movable frame accommodation portion 12c from the outside in the radial direction. An inner circumferential surface of the yoke 32 is fixed to the outer circumferential surface of the movable frame accommodation portion 12c. Thus, the yoke 32 is held by the movable frame 31. A coil accommodation portion 32a and a notch portion 32b are provided on the yoke 32.

The coil accommodation portion 32a is a hole that is recessed radially outward from the inner circumferential surface of the yoke 32. The coil accommodation portion 32a extends in the circumferential direction and is provided all around in the circumferential direction. As shown in FIG. 2, the notch portion 32b is a hole that radially passes through a portion of the yoke 32 in the circumferential direction. In this embodiment, the notch portion 32b has a substantially rectangular shape of which a long side extends in the optical axis direction when seen in the radial direction. When seen in the radial direction, a shape of the notch portion 32b may be, for example, another shape such as a circular shape. As shown in FIG. 3, the inside of the coil accommodation portion 32a and the outside of the yoke 32 are connected via the notch portion 32b.

Each of the pair of magnets 33 has a substantially annular shape centered on the optical axis J. Each of the pair of magnets surrounds the movable frame accommodation portion 12c from the outside in the radial direction. Each of the pair of magnets 33 is fixed to the outer circumferential surface of the movable frame accommodation portion 12c. Thus, each of the pair of magnets 33 is held by the holding frame 12. In the optical axis direction, the pair of magnets 33 are disposed with the yoke 32 interposed therebetween. One magnet 33 is disposed on the distal end side of the yoke 32, that is, on the other side (the +Z side) in the optical axis direction. The one magnet 33 is in contact with the distal end of the yoke 32 in the optical axis direction. The other magnet 33 is disposed on the proximal end side of the yoke 32, that is, on one side (the −Z side) in the optical axis direction. The other magnet 33 is in contact with the proximal end of the yoke 32 in the optical axis direction. The other magnet 33 is in contact with a surface of the first annular portion 12b that faces the distal end side in the optical axis direction. Each of the pair of magnets 33 has a pair of magnetic poles. The S pole of one magnet 33 faces the distal end side, and the N pole faces the proximal end side. The S pole of the other magnet 33 faces the proximal end side, and the N pole faces the distal end side. The pair of magnets 33 are disposed such that the same magnetic poles, in this embodiment, the N poles face each other in the optical axis direction. The pair of magnets 33 may be disposed with the S poles facing each other in the optical axis direction.

The coil part 34 is electrically connected to the substrate 50. A current is supplied to the coil part 34 from an external device (not shown) via the substrate 50 and the cable 60. The coil part 34 includes a coil main body portion 34a and a wiring portion 35. That is, the electronic component 30 has the wiring portion 35. In this embodiment, the coil part 34 is configured of a coil wire in which a conductive copper wire is coated with an insulation coating such as enamel.

The coil main body portion 34a is accommodated inside the coil accommodation portion 32a of the yoke 32. That is, the coil main body portion 34a is accommodated inside the yoke 32. The coil main body portion 34a is circumferentially wound around the outer circumferential surface of the movable frame accommodation portion 12c. The coil main body portion 34a is fixed to the outer circumferential surface of the movable frame accommodation portion 12c. Thus, the coil part 34 is held by the holding frame 12.

In this embodiment, the current supplied from the external device to the coil main body portion 34a via the substrate 50 and the cable 60 is controlled according to an operation of a button (not shown) of the operation part 3 or an external device (not shown). As shown in FIG. 3, when a current is supplied to the coil main body portion 34a, the coil main body portion 34a forms an electromagnet with a magnetic pole facing in the optical axis direction. More specifically, when a current flowing toward one side (+θ side) in the circumferential direction is supplied to the coil main body portion 34a, the coil main body portion 34a constitutes an electromagnet with the N pole facing the proximal end side and the S pole facing the distal end side. Thus, since a magnetic field of the other magnet 33 disposed on the proximal end side of the yoke 32 is canceled, the movable frame 31 moves to the distal end side due to a magnetic force with the one magnet 33 disposed on the distal end side of the yoke 32. When the movable frame 31 comes into contact with the second restriction surface 13e in the optical axis direction, the movement of the movable frame 31 to the distal end side is stopped.

When the supply of the current to the coil main body portion 34a is stopped in a state in which the movable frame 31 is in contact with the second restriction surface 13e in the optical axis direction, the coil main body portion 34a does not constitute an electromagnet. At this time, a portion of the movable frame 31 on the distal end side overlaps one magnet 33 in the radial direction and is located closer to the distal end side than the other magnet 33. Therefore, since the magnetic force between one magnet 33 and the movable frame 31 is larger than the magnetic force between the other magnet 33 and the movable frame 31, a magnetic force directed to the distal end side is applied to the movable frame 31. Therefore, the state in which the movable frame 31 and the second restriction surface 13e are in contact with each other in the optical axis direction is maintained.

When a current flowing to the other side (the −θ side) in the circumferential direction is supplied to the coil main body portion 34a, the coil main body portion 34a constitutes an electromagnet with the N pole facing the distal end side and the S pole facing the proximal end side. Thus, since the magnetic field of one magnet 33 disposed on the distal end side of the yoke 32 is canceled, the movable frame 31 moves to the proximal end side due to the magnetic force with the other magnet 33. When the movable frame 31 comes into contact with the first restriction surface 12e in the optical axis direction, the movement of the movable frame 31 to the proximal end side is stopped.

Although not shown, when the supply of the current to the coil main body portion 34a is stopped in a state in which the movable frame 31 is in contact with the first restriction surface 12e in the optical axis direction, the coil main body portion 34a does not constitute an electromagnet. At this time, a portion of the movable frame 31 on the proximal end side overlaps the other magnet 33 in the radial direction and is located closer to the proximal end side than the one magnet 33. Therefore, since the magnetic force between the other magnet 33 and the movable frame 31 is larger than the magnetic force between the one magnet 33 and the movable frame 31, a magnetic force directed to the proximal end side is applied to the movable frame 31. Therefore, the state in which the movable frame 31 and the first restriction surface 12e are in contact with each other in the optical axis direction is maintained. As described above, the movable lens 23 held by the movable frame 31 can be moved in the optical axis direction by appropriately switching a direction of the current supplied to the coil main body portion 34a, and thus the focal point of the subject in the endoscope 1 can be changed.

The wiring portion 35 is a portion of the coil part 34 that is pulled out of the coil main body portion 34a and connected to the substrate 50. As shown in FIG. 2, the wiring portion 35 is pulled out of the notch portion 32b of the yoke 32 to the outside of the yoke 32. The wiring portion 35 is pulled out of the notch portion 32b to the substantially proximal end side. In addition, in the following description, a portion of the end portion of the wiring portion 35 that is pulled out of the coil main body portion 34a may be referred to as one end of the wiring portion 35, and a portion of the end portion of the wiring portion 35 that is connected to the substrate 50 may be referred to as the other end of the wiring portion 35. The wiring portion 35 has a first wiring 35a and a second wiring 35b.

Although not shown, the first wiring 35a is pulled out of one side of the coil main body portion 34a. The second wiring 35b is pulled out of the other side of the coil main body portion 34a. Thus, the first wiring 35a and the second wiring 35b are connected in series via the coil main body portion 34a. Each of the first wiring 35a and the second wiring 35b is electrically connected to the substrate 50. In this embodiment, a length of the first wiring 35a is longer than a length of the second wiring 35b.

The cable 60 is electrically connected to an external device (not shown) via the connector 9 shown in FIG. 1. As shown in FIG. 2, the cable 60 is electrically connected to the substrate 50. The coil part 34 is electrically connected to an external device via the cable 60 and the substrate 50. In this embodiment, the endoscopic imaging device 10 has two cables 60.

The substrate 50 electrically connects the wiring portion 35 and the cable 60. Thus, an external device (not shown) and the electronic component 30 are electrically connected, and a current is supplied from the external device to the electronic component 30. The substrate 50 has a winding portion 52 and a straight portion 51. The winding portion 52 extends in the circumferential direction along the outer circumferential surface of the holding frame 12. More specifically, as shown in FIG. 3, the winding portion 52 extends in the circumferential direction along an outer circumferential surface of a portion of the lens accommodation portion 12a that is closer to the distal end side than the lens barrel part 11. As shown in FIG. 2, the winding portion 52 is disposed closer to one side (the −Z side) than the electronic component 30 in the optical axis direction. That is, the electronic component 30 is disposed closer to the other side (the +Z side) than the winding portion 52 in the optical axis direction. In this embodiment, the winding portion 52 extends from the distal end portion of the straight portion 51 to the other side (the −θ side) in the circumferential direction. The winding portion 52 may extend from the distal end portion of the straight portion 51 to one side (the +θ side) in the circumferential direction. In this embodiment, the winding portion 52 is fixed to the outer circumferential surface of the lens accommodation portion 12a with a double-sided tape, an adhesive, or the like. That is, the winding portion 52 is fixed to the holding frame 12. In this embodiment, an end portion of the winding portion 52 on one side in the circumferential direction and an end portion thereof on the other side in the circumferential direction are disposed with a slight interval in the circumferential direction. The end portion of the winding portion 52 on one side in the circumferential direction and the end portion thereof on the other side in the circumferential direction may be in contact with each other in the circumferential direction.

The straight portion 51 extends from the winding portion 52 to the proximal end side, that is, to one side (the −Z side) in the optical axis direction. More specifically, the straight portion 51 extends linearly from an edge portion of the winding portion 52 on one side (the +θ side) in the circumferential direction to the proximal end side. The straight portion 51 is disposed across the holding frame 12 and the lens barrel part 11. In the optical axis direction, an end portion of the straight portion 51 on the proximal end side is located at a portion of the lens barrel part 11 on the proximal end side. The straight portion 51 may be disposed across the reinforcing frame 15. An edge portion of the straight portion 51 on the proximal end side is connected to the cable 60. A portion of the straight portion 51 on the proximal end side is disposed inside the substrate mounting portion 11a of the lens barrel part 11. The portion of the straight portion 51 on the proximal end side is fixed to an inner surface of the substrate mounting portion 11a with a double-sided tape, an adhesive, or the like. That is, a portion of the straight portion 51 is fixed to the outer circumferential surface of the lens barrel part 11. Thus, a position of the straight portion 51 in the circumferential direction with respect to the lens barrel part 11 is determined. A portion of the straight portion 51 on the distal end side is fixed to the outer circumferential surface of the holding frame 12 with a double-sided tape, an adhesive, or the like.

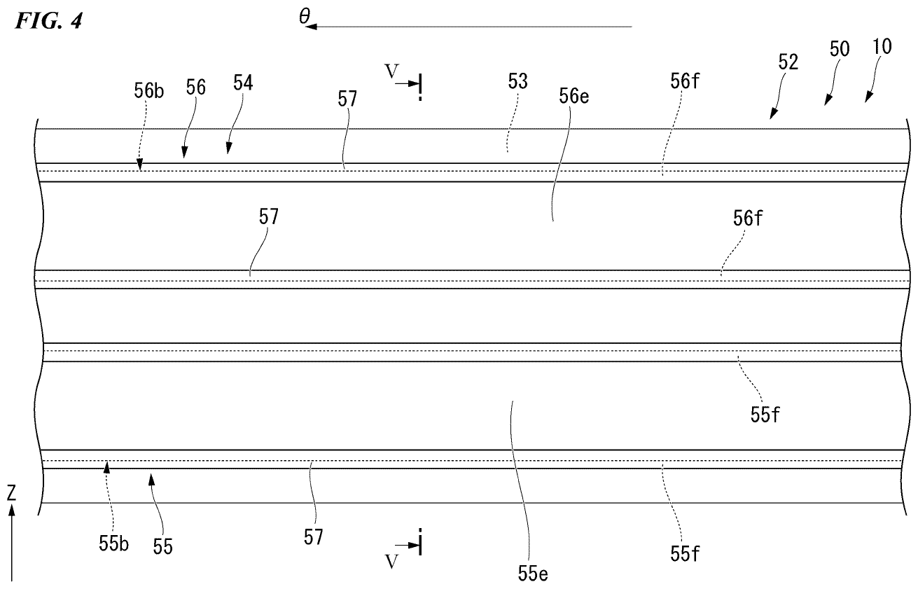

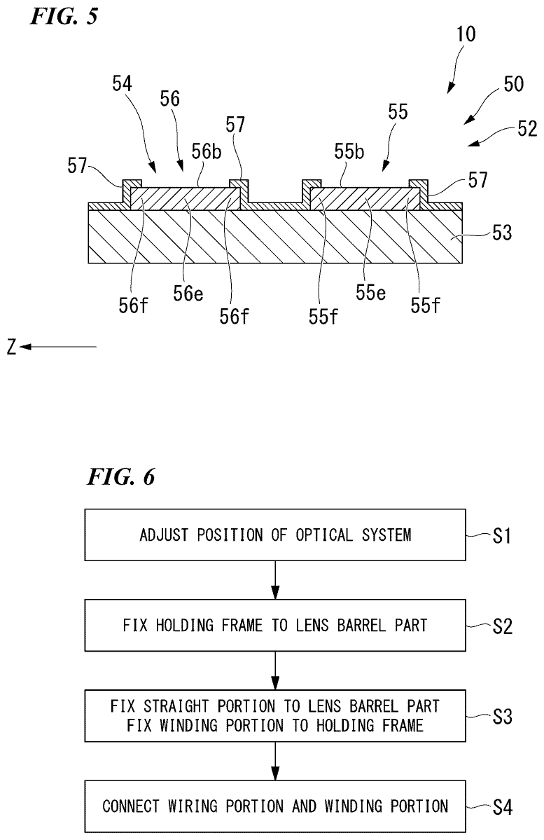

FIG. 4 is a side view showing a part of the winding portion 52 of this embodiment. FIG. 5 is a cross-sectional view showing the winding portion 52 of this embodiment, and is a cross-sectional view taken along line V-V in FIG. 4.

In this embodiment, the substrate 50 is a flexible print circuit that is flexible in a thickness direction. As shown in FIG. 5, the substrate 50 includes a base layer 53, a conductive portion 54, and a coating layer 57. In this embodiment, the substrate 50 is covered with an insulating resin film (not shown). Thus, the conductive portion 54 and the subject or the like can be insulated.

The base layer 53 is a resin film that extends in a direction perpendicular to the radial direction. The base layer 53 is made of, for example, an insulating resin material such as polyimide and polyester. In this embodiment, the base layer 53 is made of polyimide. From the viewpoint of strength and flexibility of the substrate 50, a thickness of the base layer 53 is preferably 10 μm or more and 50 μm or less.

The conductive portion 54 is formed on the base layer 53. The conductive portion 54 extends in a direction perpendicular to the radial direction. The conductive portion 54 has conductivity. In this embodiment, the conductive portion 54 is a copper foil. From the viewpoint of electrical resistance of the conductive portion 54, a thickness of the conductive portion 54 is preferably 10 μm or more. In this embodiment, the conductive portion 54 and the base layer 53 are fixed to each other with an adhesive (not shown). As the adhesive, for example, a thermosetting resin such as an epoxy resin and an acrylic resin can be used. As shown in FIG. 2, in this embodiment, the conductive portion 54 is disposed across the winding portion 52 and the straight portion 51. The conductive portion 54 includes a first conductive portion 55 and a second conductive portion 56.

The first conductive portion 55 is connected to the first wiring 35a. The first conductive portion 55 has a first extending portion 55a and a first curved portion 55b. The first extending portion 55a is a portion of the first conductive portion 55 that is disposed on the straight portion 51. The first extending portion 55a extends in the optical axis direction. The first extending portion 55a extends from an edge portion of the straight portion 51 on the proximal end side to an edge portion thereof on the distal end side.

The first curved portion 55b is a portion of the first conductive portion 55 that is disposed on the winding portion 52. The first curved portion 55b extends in the circumferential direction. That is, in the winding portion 52, the first conductive portion 55 extends in the circumferential direction. The first curved portion 55b extends from an edge portion of the winding portion 52 on the one side (the +θ side) in the circumferential direction to an edge portion thereof on the other side (the −θ side) in the circumferential direction. An end portion of the first curved portion 55b in the circumferential direction is connected to the distal end of the first extending portion 55a.

The second conductive portion 56 is connected to the second wiring 35b. The second conductive portion 56 includes a second extending portion 56a and a second curved portion 56b. The second extending portion 56a is a portion of the second conductive portion 56 that is disposed on the straight portion 51. The second extending portion 56a extends in the optical axis direction. The second extending portion 56a extends from the edge portion of the straight portion 51 on the proximal end side to the edge portion thereof on the distal end side. The second extending portion 56a is disposed closer to one side (the +θ side) in the circumferential direction than the first extending portion 55a.

The second curved portion 56b is a portion of the second conductive portion 56 that is disposed on the winding portion 52. The second curved portion 56b extends in the circumferential direction. That is, the second conductive portion 56 in the winding portion 52 extends in the circumferential direction. The second curved portion 56b extends from an edge portion on one side (the +θ side) of the winding portion 52 in the circumferential direction to an edge portion on the other side (the −θ side) in the circumferential direction. The end portion of the second curved portion 56b on one side in the circumferential direction is connected to the distal end of the second extending portion 56a. The second curved portion 56b is disposed closer to the distal end side than the first curved portion 55b. That is, in the winding portion 52, the first conductive portion 55 is disposed closer to the proximal end side than the second conductive portion 56, that is, on one side (the −Z side) in the optical axis direction.

As shown in FIG. 5, the coating layer 57 is formed across the base layer 53 and the conductive portion 54. The coating layer 57 covers a portion of the conductive portion 54 from the outside in the radial direction. Thus, peeling of the conductive portion 54 from the base layer 53 can be curbed. The coating layer 57 is made of, for example, an insulating resin material such as polyimide and polyester.

As shown in FIGS. 4 and 5, the first curved portion 55b has an exposed portion 55e exposed to the outside in the radial direction, and a coated portion 55f covered with the coating layer 57 from the outside in the radial direction. The second curved portion 56b has an exposed portion 56e and a coated portion 56f. That is, in the winding portion 52, the conductive portion 54 has the exposed portions 55e and 56e and coated portions 55f and 56f. In this embodiment, as shown in FIG. 4, the coating layer 57 extends in the circumferential direction along an edge portion of each of the first curved portion 55b and the second curved portion 56b on the distal end side and an edge portion thereof on the proximal end side. Therefore, each of the coated portions 55f and 56f extends in the circumferential direction. As shown in FIG. 5, in this embodiment, each of the exposed portions 55e and 56e is a portion of each of the first curved portion 55b and the second curved portion 56b on the center side in the optical axis direction. As shown in FIG. 4, the exposed portions 55e and 56e extend in the circumferential direction. Although not shown, the exposed portion 55e extends from an end portion of the first curved portion 55b on one side (the +θ side) in the circumferential direction to an end portion thereof on the other side (the −θ side) in the circumferential direction. The exposed portion 56e extends from an end portion of the second curved portion 56b on the one side in the circumferential direction to an end portion thereof on the other side in the circumferential direction.

Although not shown, the first wiring 35a is connected to the exposed portion 55e of the first curved portion 55b, and the second wiring 35b is connected to the exposed portion 56e of the second curved portion 56b. Thus, the first wiring 35a is connected to the first conductive portion 55, and the second wiring 35b is connected to the second conductive portion 56. That is, the wiring portion 35 is connected to the conductive portion 54 disposed on the winding portion 52.

The first wiring 35a is connected to a portion of the first conductive portion 55 near the notch portion 32b. The second wiring 35b is connected to a portion of the second conductive portion 56 near the notch portion 32b. That is, the wiring portion 35 is connected to a portion of the conductive portion 54 near a position at which the wiring portion 35 is pulled out of the electronic component 30. The position at which the wiring portion 35 and the conductive portion 54 are connected may overlap the notch portion 32b when seen in the optical axis direction, or may be slightly shifted from the notch portion 32b in the circumferential direction. In this embodiment, each of the first wiring 35a and the second wiring 35b is connected to each of the first curved portion 55b and the second curved portion 56b by soldering.

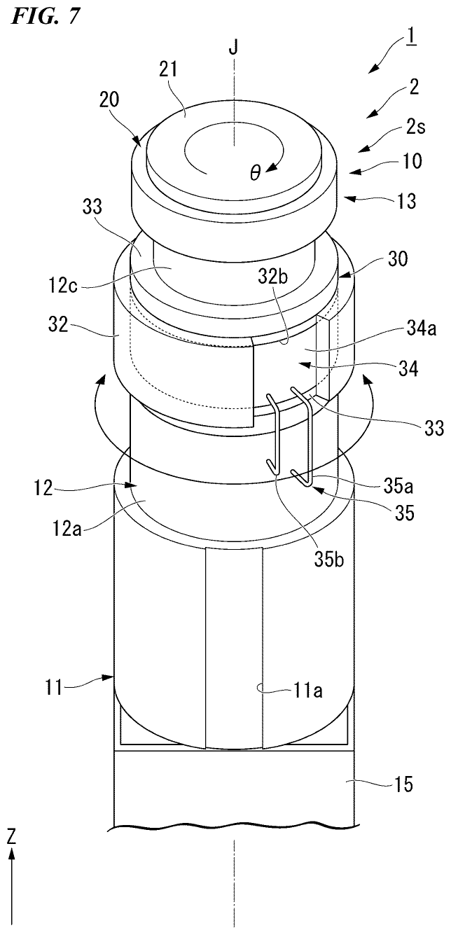

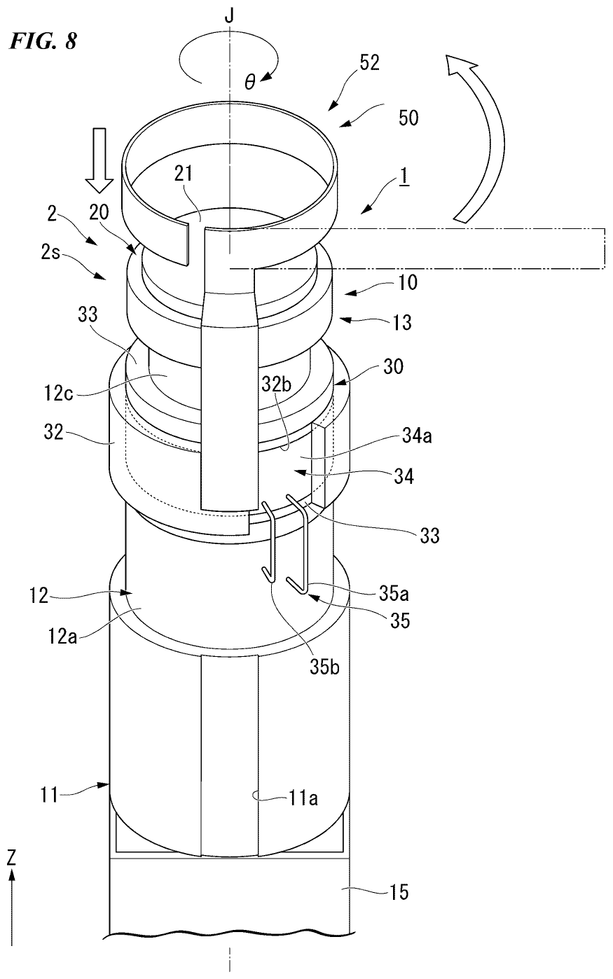

FIG. 6 is a flowchart showing a method for manufacturing the endoscopic imaging device 10 of this embodiment. FIG. 7 is a first perspective view showing an assembly process of the endoscopic imaging device 10 of this embodiment. FIG. 8 is a second perspective view showing an assembly process of the endoscopic imaging device 10 of this embodiment.

The method for manufacturing the endoscopic imaging device 10 of this embodiment includes a first step S1 of adjusting a position of the optical system 20 by moving the holding frame 12 in the optical axis direction and rotating it in the circumferential direction with respect to the lens barrel part 11, a second step S2 of fixing the holding frame 12 to the lens barrel part 11, a third step of fixing the straight portion 51 to the lens barrel part 11 and fixing the winding portion 52 to the holding frame 12, and a fourth step S4 of connecting the wiring portion 35 and the winding portion 52. In the following description, an “operator or the like” includes the operator who performs each work, assembly equipment, and the like. Each work may be performed only by an operator, only by an assembly device, or by the operator and an assembly device.

In the first step S1, the operator or the like adjusts the position of the optical system 20 by moving the holding frame 12 in the optical axis direction and rotating it in the circumferential direction with respect to the lens barrel part 11. As shown in FIG. 7, the operator or the like moves the holding frame 12, on which the optical system 20, the objective lens holding part 13, the electronic component 30, and the like are mounted in advance, from the distal end side of the lens barrel part 11 fixed to a jig or the like to the proximal end side, and inserts the holding frame 12 into the inside of the lens barrel part 11 through an opening of the lens barrel part 11 on the distal end side. Next, the operator or the like moves the holding frame 12 in the optical axis direction and rotates it in the circumferential direction with respect to the lens barrel part 11 to adjust the position of each of the lens constituting the optical system 20 in the optical axis direction and the circumferential direction. Thus, a position of a focal point of the optical image of the subject formed by the optical system 20 can be made to coincide with a light receiving surface of the imaging element 29 shown in FIG. 4, and an inclination of the optical axis can be curbed. At this time, since the shape and arrangement of each of the lens constituting the optical system 20 differ according to the endoscopic imaging device 10, a position of the holding frame 12 in the circumferential direction with respect to the lens barrel part 11 differs according to the endoscopic imaging device 10. Therefore, a position of the notch portion 32b of the electronic component 30 in the circumferential direction with respect to the substrate mounting portion 11a of the lens barrel part 11 differs according to the endoscopic imaging device 10. When the adjustment of the position of the optical system 20 is completed, the first step S1 is completed. In the second step S2, the operator or the like fixes the holding frame 12 to the lens barrel part 11. In the first step S1, the operator or the like fixes the holding frame 12 in which the position of the optical system 20 has been adjusted and the lens barrel part 11 with an adhesive (not shown). As the adhesive, an epoxy adhesive, a urethane adhesive, or the like can be used. When the holding frame 12 is fixed to the lens barrel part 11, the second step S2 is completed.

In the third step S3, the operator or the like fixes the straight portion 51 to the lens barrel part 11 and fixes the winding portion 52 to the holding frame 12. First, the operator or the like moves the substrate 50 from the distal end side of the holding frame 12 or the like to the proximal end side, and passes the objective lens holding part 13 and the holding frame 12 inside the winding portion 52 in the optical axis direction. At this time, the operator or the like adjusts the position of the substrate 50 in the circumferential direction with respect to the lens barrel part 11 so that the straight portion 51 is disposed inside the substrate mounting portion 11a of the lens barrel part 11.

Next, when the substrate 50 is moved to the proximal end side until the winding portion 52 faces the outer circumferential surface of the lens accommodation portion 12a of the holding frame 12, the operator or the like fixes the winding portion 52 and a portion of the straight portion 51 on the distal end side to the outer circumferential surface of the lens accommodation portion 12a with a double-sided tape, an adhesive, or the like, and fixes a portion of the straight portion 51 on the proximal end side to an inner surface of the substrate mounting portion 11a with a double-sided tape, an adhesive, or the like. Thus, as shown in FIGS. 2 and 3, the substrate 50 is fixed to the holding frame 12 and the lens barrel part 11. As described above, since the position of the notch portion 32b of the electronic component 30 in the circumferential direction with respect to the substrate mounting portion 11a differs according to the endoscopic imaging device 10, the position of the wiring portion 35 in the circumferential direction with respect to the straight portion 51 differs according to the endoscopic imaging device 10. Once the substrate 50 is fixed to the holding frame 12 and the lens barrel part 11, the third step S3 is completed. To simplify the assembly, the winding portion 52 of which a shape is maintained in advance according to a shape of the outer circumferential surface of the lens accommodation portion 12a may be fixed to the outer circumferential surface of the lens accommodation portion 12a.

In the fourth step S4, the operator or the like connects the wiring portion 35 and the winding portion 52. As shown in FIG. 2, the operator or the like pulls out the first wiring 35a from the notch portion 32b to the proximal end side, and connects the other end of the first wiring 35a to the exposed portion 55e of the first curved portion 55b shown in FIG. 4 by soldering. Next, as shown in FIG. 2, the operator or the like pulls out the second wiring 35b from the notch portion 32b to the proximal end side, and connects the other end of the second wiring 35b to the exposed portion 56e of the second curved portion 56b shown in FIG. 4 by soldering. Thus, the wiring portion 35 is connected to a portion of the conductive portion 54 near a position at which the wiring portion 35 is pulled out of the electronic component 30. Further, the first wiring 35a is electrically connected to the first conductive portion 55, and the second wiring 35b is electrically connected to the second conductive portion 56. That is, the wiring portion 35 is electrically connected to the conductive portion 54. Next, the operator or the like connects the cable 60 to the end portion of each of the first extending portion 55a and the second extending portion 56a on the proximal end side by soldering. Thus, the electronic component 30 and the cable 60 are electrically connected via the substrate 50. When the cable 60 is connected to the substrate 50, the fourth step S4 is completed. When the fourth step S4 is completed, the assembly process of the endoscopic imaging device 10 is completed.

According to this embodiment, the endoscopic imaging device 10 includes the optical system 20 that forms an optical image, the tubular holding frame 12 that accommodates a part of the optical system 20 therein and extends in the optical axis direction, the electronic component 30 having the wiring portion 35 and held in the holding frame 12, and the substrate 50 having the winding portion 52 that extends in the circumferential direction along the outer circumferential surface of the holding frame 12, and the straight portion 51 that extends from the winding portion 52 to the proximal end side, that is, one side (the −Z side) in the optical axis direction. The substrate 50 has the conductive portion 54 disposed across the winding portion 52 and the straight portion 51, and the wiring portion 35 is connected to the conductive portion 54 disposed in the winding portion 52. In the case of a configuration in which the substrate 50 has only the straight portion 51, when the position in the circumferential direction at which the wiring portion 35 is pulled out of the electronic component 30 with respect to the straight portion 51 varies according to the endoscopic imaging device 10 by adjusting the position of the optical system 20 in the first step S1 of the assembly process of the endoscopic imaging device 10, a shortest distance between one end of the wiring portion 35 and the substrate 50 varies according to the endoscopic imaging device 10. Therefore, in the fourth step S4, a work of adjusting the length of the wiring portion 35 and a work of removing the insulation coating of the wiring portion 35 are required, and thus the number of assembly steps for the endoscopic imaging device 10 increases. Further, it is necessary to pull out the wiring portion 35 not only in the optical axis direction but also in the circumferential direction. Therefore, when the wiring portion 35 is pulled out toward the straight portion 51, the wiring portion 35 tends to get caught on the inner surface of the notch portion 32b, the outer circumferential surface of the holding frame 12, or the like, and thus there is a possibility that the wiring portion 35 will be disconnected.

On the other hand, in this embodiment, since the wiring portion 35 is connected to the winding portion 52 that extends in the circumferential direction along the outer circumferential surface of the holding frame 12, even when the position in the circumferential direction at which the wiring portion 35 is pulled out of the electronic component 30 with respect to the straight portion 51 varies according to the endoscopic imaging device 10, it is possible to curb a change in the shortest distance between one end of the wiring portion 35 and the substrate 50. Therefore, since the length of the wiring portion 35 can be made the same in all endoscopic imaging devices 10, in the fourth step S4 of the assembly process of the endoscopic imaging device 10, there is no need to adjust the length of the wiring portion 35 according to the endoscopic imaging device 10, and also, in a process prior to the assembly process of the endoscopic imaging device 10, the insulation coating of the wiring portion 35 can be removed in advance. Therefore, in the fourth step S4, since the work of adjusting the length of the wiring portion 35 and the work of removing the insulation coating of the wiring portion 35 are no longer necessary, it is possible to curb an increase in the number of assembly steps for the endoscopic imaging device 10. Furthermore, since the wiring portion 35 can be connected to the winding portion 52 by pulling out the wiring portion 35 only in the substantially optical axis direction, when the wiring portion 35 is pulled out, it is possible to curb the wiring portion 35 getting caught on the inner surface of the notch portion 32b, the outer circumferential surface of the holding frame 12, or the like. Therefore, disconnection of the wiring portion 35 can be curbed.

Furthermore, in this embodiment, since a change in the length of the wiring portion 35 according to the endoscopic imaging device 10 can be curbed, a change in an electrical resistance value of the wiring portion 35 can be curbed. Therefore, since a current value supplied to the coil main body portion 34a can be stabilized, the movable frame 31 can be stably moved in the optical axis direction. Therefore, since the movable lens 23 held by the movable frame 31 can be stably moved in the optical axis direction, the focal point of the subject in the endoscope 1 can be stably changed.

Further, in this embodiment, as described above, in the fourth step S4 of the assembly process of the endoscopic imaging device 10, since disconnection of the wiring portion 35 can be curbed, a diameter of the wiring portion 35 can be reduced. Therefore, even without providing a groove in the holding frame 12 for passing the wiring portion 35, it is possible to curb an increase in a dimension of the endoscopic imaging device 10 in the radial direction. Therefore, it is possible to curb an increase in the number of assembly steps for the holding frame 12, and thus, it is possible to curb an increase in the number of assembly steps for the endoscopic imaging device 10.

According to this embodiment, the wiring portion 35 is connected to a portion of the conductive portion 54 near the position at which the wiring portion 35 is pulled out of the electronic component 30. Therefore, a change in the distance between one end of the wiring portion 35 and the conductive portion 54 can be curbed more appropriately. Therefore, in the fourth step S4 of the assembly process of the endoscopic imaging device 10, since the work of adjusting the length of the wiring portion 35 and the work of removing the insulation coating of the wiring portion 35 are no longer necessary, it is possible to curb an increase in the number of assembly steps for the endoscopic imaging device 10.

Further, in this embodiment, the wiring portion 35 may be connected by soldering to a portion of the winding portion 52 that is shifted in the circumferential direction from the position at which the wiring portion 35 is pulled out of the electronic component 30. In this case, the length of the wiring portion 35 can be made longer than the shortest distance from one end of the wiring portion 35 to the winding portion 52. Therefore, even if a copper wire of the wiring portion 35 becomes short due to copper corrosion when the wiring portion 35 is soldered to the winding portion 52, the wiring portion 35 can be connected again to a portion of the winding portion 52 that overlaps in the optical axis direction the position at which the wiring portion 35 is pulled out of the electronic component 30. Therefore, even when the copper wire of the wiring portion 35 is shortened due to copper corrosion, the coil part 34 can be connected to the substrate 50 without replacing the coil part 34, and thus it is possible to curb an increase in the number of assembly steps and manufacturing cost of the endoscopic imaging device 10.

According to this embodiment, the electronic component 30 includes the movable frame 31 that is disposed inside the holding frame 12 and holds a part of the optical system 20 therein, the yoke 32 that is provided outside the holding frame 12, the pair of magnets 33 in which the same magnetic poles are disposed to face each other in the optical axis direction on the proximal end side of the yoke 32, that is, one side (the −Z side) in the optical axis direction, and the distal end side, that is, the other side (the +Z side) in the optical axis direction, and the coil main body portion 34a that is accommodated inside the yoke 32, and the movable frame 31 is made of a magnetic body and is movable in the optical axis direction. Therefore, as described above, since the magnetic field of either one of the pair of magnets 33 can be canceled by controlling the direction of the current supplied to the coil main body portion 34a, the movable frame 31 can be moved in the optical axis direction. Thus, the focal point of the subject in the endoscope 1 can be changed, and thus the endoscopic imaging device 10 can capture a clearer image of the subject.

According to this embodiment, the yoke 32 is provided with the notch portion 32b, and the wiring portion 35 is pulled out from the notch portion 32b to the outside of the yoke 32. Therefore, in the fourth step S4 of the assembly process of the endoscopic imaging device 10, the wiring portion 35 connected to the coil main body portion 34a accommodated inside the yoke 32 can be easily pulled out to the outside of the yoke 32. Therefore, it is possible to simplify the work of connecting the wiring portion 35 to the substrate 50, and thus it is possible to curb an increase in the number of assembly steps for the endoscopic imaging device 10.

According to this embodiment, the endoscopic imaging device 10 includes the tubular lens barrel part 11 that extends in the optical axis direction, the lens barrel part 11 holds the holding frame 12, a part of the straight portion 51 is fixed to the outer circumferential surface of the lens barrel part 11, and the winding portion 52 is fixed to the holding frame 12. Therefore, since the winding portion 52 is fixed to the holding frame 12, a change in the position of the winding portion 52 with respect to the electronic component 30 can be curbed. Therefore, in the fourth step S4 of the assembly process of the endoscopic imaging device 10, since the wiring portion 35 can be more easily connected to the winding portion 52, it is possible to curb an increase in the number of assembly steps for the endoscopic imaging device 10. Moreover, since it is possible to curb a change in the position of the winding portion 52 with respect to the electronic component 30 after the endoscopic imaging device 10 is assembled, the connection between the wiring portion 35 and the substrate 50 can be stabilized. Therefore, the endoscopic imaging device 10 can be stably operated.

Furthermore, in this embodiment, since the lens barrel part 11 holds the holding frame 12 and the straight portion 51 is fixed to the lens barrel part 11, the position of the straight portion 51 with respect to the holding frame 12 can be determined via the lens barrel part 11. Thus, since a change in the position of the winding portion 52 with respect to the electronic component 30 can be more effectively curbed, the wiring portion 35 can be more easily connected to the substrate 50 in the fourth step S4 of the assembly process of the endoscopic imaging device 10. Therefore, it is possible to curb an increase in the number of assembly steps for the endoscopic imaging device 10.

According to this embodiment, the substrate 50 includes the conductive portion 54 having conductivity and the coating layer 57 that covers a part of the conductive portion 54, in the winding portion 52, the conductive portion 54 has the exposed portions 55e and 56e that are exposed to the outside in the radial direction, and coated portions 55f and 56f that are covered with the coating layer 57, and the exposed portions 55e and 56e extend in the circumferential direction. Therefore, in the circumferential direction, the degree of freedom in a position at which the wiring portion 35 is connected to the conductive portion 54 can be increased. Therefore, in the fourth step S4 of the assembly process of the endoscopic imaging device 10, since the wiring portion 35 can be more easily connected to the conductive portion 54, it is possible to curb an increase in the number of assembly steps for the endoscopic imaging device 10.

According to this embodiment, the wiring portion 35 includes the first wiring 35a and the second wiring 35b, the length of the first wiring 35a is longer than the length of the second wiring 35b, the electronic component 30 is disposed closer to the distal end side than the winding portion 52, that is, on the other side (the +Z side) in the optical axis direction, the conductive portion 54 includes the first conductive portion 55 and the second conductive portion 56, in the winding portion 52, each of the first conductive portion 55 and the second conductive portion 56 extends in the circumferential direction, also the first conductive portion 55 is disposed closer to the proximal end side than the second conductive portion 56, that is, on one side (the −Z side) in the optical axis direction, the first wiring 35a is connected to the first conductive portion 55, and the second wiring 35b is connected to the second conductive portion 56. Therefore, as shown in FIG. 2, a distance in the optical axis direction between one end of the first wiring 35a and the first curved portion 55b of the first conductive portion 55 is longer than a distance in the optical axis direction between one end of the second wiring 35b and the second curved portion 56b of the second conductive portion 56. Therefore, in the fourth step of the assembly process of the endoscopic imaging device 10, it is easy to curb the first wiring 35a, which is longer than the second wiring 35b, being mistakenly connected to the second curved portion 56b, and it is easy to curb the second wiring 35b, which is shorter than the first wiring 35a, being mistakenly connected to the first curved portion 55b. Therefore, since the first wiring 35a can be easily connected to the first conductive portion 55, and the second wiring 35b can be easily connected to the second conductive portion 56, it is possible to more appropriately curb an increase in the number of assembly steps for the endoscopic imaging device 10.

Further, in this embodiment, by making the length of the second wiring 35b shorter than the shortest distance between one end of the second wiring 35b and the first curved portion 55b, it is possible to more preferably curb the second wiring 35b being mistakenly connected to the first curved portion 55b. Thus, it is possible to more preferably curb an increase in the number of assembly steps for the endoscopic imaging device 10.

According to this embodiment, the endoscope 1 includes the inserting part 2 inserted into a subject, and the endoscopic imaging device 10 provided at the distal end of the inserting part 2. The endoscopic imaging device 10 includes the optical system 20 that forms an optical image, the electronic component 30 having the wiring portion 35, the tubular holding frame 12 that accommodates a part of the optical system 20 therein and extends in the optical axis direction, and the substrate 50 having the winding portion 52 that extends in the circumferential direction along the outer circumferential surface of the holding frame 12, and the straight portion 51 that extends from the winding portion 52 to the proximal end side, that is, to one side (the −Z side) in the optical axis direction, and the wiring portion 35 is connected to a portion of the winding portion 52 near a position at which the wiring portion 35 is pulled out of the electronic component 30. Therefore, even when the position in the circumferential direction at which the wiring portion 35 is pulled out of the electronic component 30 with respect to the straight portion 51 varies according to the endoscopic imaging device 10, since the wiring portion 35 is connected to the winding portion 52 that extends in the circumferential direction along the outer circumferential surface of the holding frame 12, it is possible to curb a change in the shortest distance between one end of the wiring portion 35 and the substrate 50. Therefore, in the fourth step S4 of the assembly process of the endoscopic imaging device 10, since the work of adjusting the length of the wiring portion 35 and the work of removing the insulation coating of the wiring portion 35 are no longer necessary, it is possible to curb increases in the number of assembly steps for the endoscopic imaging device 10 and the number of assembly steps for the endoscope 1.

According to this embodiment, the method for manufacturing the endoscopic imaging device 10 includes the first step S1 of adjusting the position of the optical system 20 by rotating the holding frame 12 in the circumferential direction with respect to the lens barrel part 11, the second step S2 of fixing the holding frame 12 to the lens barrel part 11, the third step S3 of fixing the straight portion 51 to the lens barrel part 11 and fixing the winding portion 52 to the holding frame 12, and the fourth step S4 of connecting the wiring portion 35 to the winding portion 52. Therefore, in the first step S1, since the holding frame 12 is rotated in the circumferential direction with respect to the lens barrel part 11, the position in the circumferential direction at which the wiring portion 35 is pulled out of the electronic component 30 with respect to the straight portion 51 fixed to the lens barrel part 11 varies according to the endoscopic imaging device 10. On the other hand, in this embodiment, in the fourth step S4, since the wiring portion 35 is connected to the winding portion 52 that extends in the circumferential direction along the outer circumferential surface of the holding frame 12, it is possible to curb a change in the shortest distance between one end of the wiring portion 35 and the substrate 50. Therefore, in the fourth step S4, since the work of adjusting the length of the wiring portion 35 and the work of removing the insulation coating of the wiring portion 35 are no longer necessary, it is possible to curb an increase in the number of assembly steps for the endoscopic imaging device 10.

Furthermore, in this embodiment, since the holding frame 12 is fixed to the lens barrel part 11 in the second step S2, it is possible to curb the change in the position of the optical system 20 after the endoscopic imaging device 10 is assembled. Therefore, since it is possible to curb the change in the position of the focal point of the optical image of the subject formed by the optical system 20, a clear image of the subject can be stably captured by the endoscopic imaging device 10.

Furthermore, in this embodiment, in the third step S3, since the straight portion 51 is fixed to the lens barrel part 11 and the winding portion 52 is fixed to the holding frame 12, it is possible to curb a change of the position of the substrate 50 with respect to the lens barrel part 11 and the holding frame 12. Thus, since the change in the position of the substrate 50 with respect to one end of the wiring portion 35 can be curbed, the wiring portion 35 and the substrate 50 can be stably connected. Therefore, the endoscopic imaging device 10 can be operated more stably.

### First Modified Example

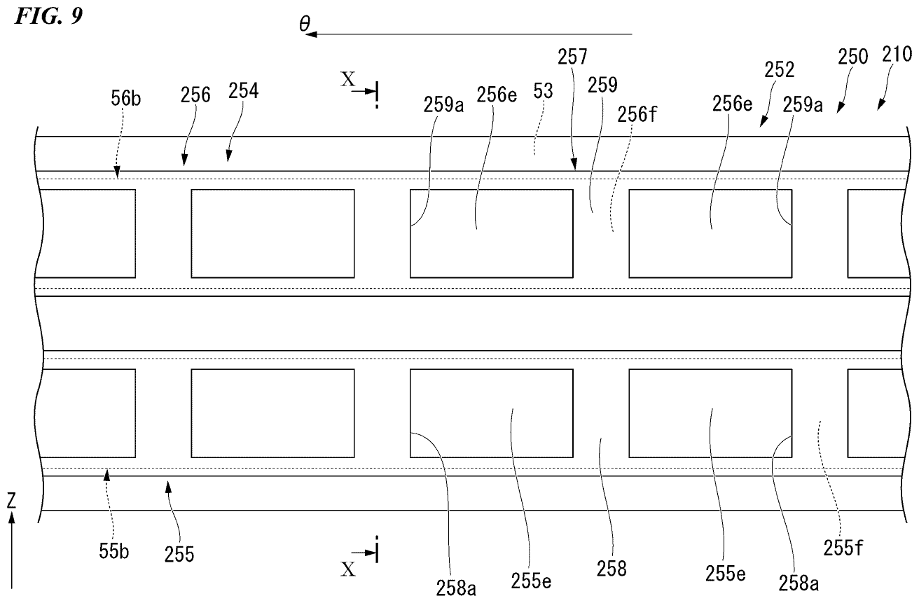

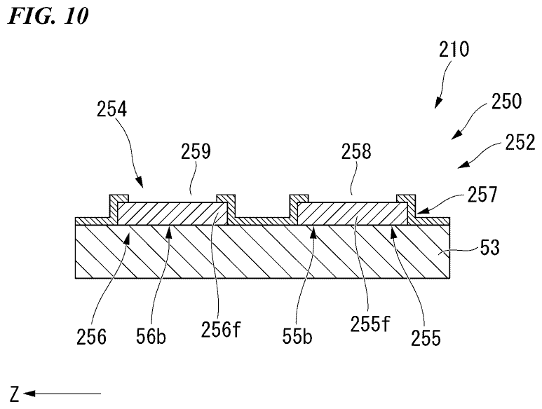

FIG. 9 is a side view showing a part of a winding portion 252 of an endoscopic imaging device 210 according to a first modified example of the first embodiment. FIG. 10 is a cross-sectional view showing the winding portion 252 of the endoscopic imaging device 210 of the first modified example of the first embodiment, and is a cross-sectional view taken along line X-X in FIG. 9. In the following description, the same reference numerals are given to the same components as in the first embodiment described above, and the description thereof will be omitted.

As shown in FIGS. 9 and 10, in this modified example, a substrate 250 includes a base layer 53, a conductive portion 254, and a coating layer 257. Configurations and the like of the base layer 53 in this modified example are the same as those of the base layer 53 in the first embodiment described above.

The conductive portion 254 is formed on the base layer 53. The conductive portion 254 has conductivity. The conductive portion 254 includes a first conductive portion 255 and a second conductive portion 256. Other configurations and the like of the conductive portion 254 are the same as those of the conductive portion 54 of the first embodiment described above.

Although not shown, the first conductive portion 255 is connected to the first wiring 35a. The second conductive portion 256 is connected to the second wiring 35b. The first conductive portion 255 includes a first extending portion 55a and a first curved portion 55b which are not shown. The second conductive portion 256 includes a second extending portion 56a and a second curved portion 56b which are not shown. The configurations and the like of the first extending portion 55a, the first curved portion 55b, the second extending portion 56a, and the second curved portion 56b are the same as those of the first extending portion 55a, the first curved portion 55b, the second extending portion 56a, and the second curved portion 56b of the first embodiment described above.

As shown in FIG. 10, the coating layer 257 is formed across the base layer 53 and the conductive portion 254. As shown in FIG. 9, the coating layer 257 covers a part of the conductive portion 254 from the outside in the radial direction. In the winding portion 252, the coating layer 257 includes a first coating layer 258 that covers a part of the first curved portion 55b from the outside in the radial direction, and a second coating layer 259 that covers a part of the second curved portion 56b from the outside in the radial direction. Other configurations and the like of the coating layer 257 are the same as those of the coating layer 57 of the first embodiment described above.

When seen in the radial direction, the first coating layer 258 has a ladder shape that extends in the circumferential direction. As shown in FIG. 10, an end portion of the first coating layer 258 on the distal end side is located closer to the distal end side than the first curved portion 55b and is fixed to the base layer 53. An end portion of the first coating layer 258 on the proximal end side is located closer to the proximal end side than the first curved portion 55b and is fixed to the base layer 53. As shown in FIG. 9, a hole 258a that passes through the first coating layer 258 in the radial direction is provided in the first coating layer 258. When seen in the radial direction, the hole 258a has a rectangular shape with long sides extending in the circumferential direction. The shape of the hole 258a may be other shapes such as a circular shape. A plurality of holes 258a are provided in the first coating layer 258. The holes 258a are disposed at intervals in the circumferential direction. When seen in the radial direction, each of the holes 258a is disposed to overlap the first curved portion 55b. As shown in FIG. 10, a portion of the first coating layer 258 in which the hole 258a is not provided in the circumferential direction is fixed to the first curved portion 55b from the end portion of the first curved portion 55b on the distal end side to the end portion thereof on the proximal end side.

As shown in FIG. 9, when seen in the radial direction, the second coating layer 259 has a ladder shape that extends in the circumferential direction. As shown in FIG. 10, an end portion of the second coating layer 259 on the distal end side is located closer to the distal end side than the second curved portion 56b and is fixed to the base layer 53. An end portion of the second coating layer 259 on the proximal end side is located closer to the proximal end side than the second curved portion 56b and is fixed to the base layer 53. As shown in FIG. 9, a hole 259a that passes through the second coating layer 259 in the radial direction is provided in the second coating layer 259. When seen in the radial direction, the hole 259a has a rectangular shape with long sides extending in the circumferential direction. The shape of the hole 259a may be other shapes such as a circular shape. In this embodiment, the shape of the hole 259a is the same as the shape of the hole 258a when seen in the radial direction. A plurality of holes 259a are provided in the second coating layer 259. The holes 259a are disposed at intervals in the circumferential direction. When seen in the radial direction, each of the holes 259a is disposed to overlap the second curved portion 56b. In this embodiment, each of the holes 259a overlaps each of the holes 258a when seen in the optical axis direction. As shown in FIG. 10, a portion of the second coating layer 259 in which the hole 259a is not provided in the circumferential direction is fixed to the second curved portion 56b from the end portion of the second curved portion 56b on the distal end side to the end portion thereof on the proximal end side.

As shown in FIGS. 9 and 10, the first curved portion 55b includes a plurality of exposed portions 255e exposed to the outside in the radial direction, and a coated portion 255f covered with the first coating layer 258 from the outside in the radial direction. The second curved portion 56b includes a plurality of exposed portions 256e that are exposed to the outside in the radial direction, and a coated portion 256f that is covered with the second coating layer 259 from the outside in the radial direction. That is, in the winding portion 252, the conductive portion 254 has the plurality of exposed portions 255e and 256e, and coated portions 255f and 256f covered with the coating layer 257.

As shown in FIG. 9, each of the plurality of exposed portions 255e is a portion of the first curved portion 55b that overlaps the plurality of holes 258a in the radial direction. Each of the plurality of exposed portions 256e is a portion of the second curved portion 56b that overlaps the plurality of holes 259a in the radial direction. When seen in the radial direction, each of the exposed portions 255e and 256e has a rectangular shape with long sides extending in the circumferential direction. The exposed portions 255e are disposed at intervals in the circumferential direction. In the circumferential direction, the exposed portions 255e are disposed with the coated portion 255f interposed therebetween. The exposed portions 256e are disposed at intervals from each other in the circumferential direction. In the circumferential direction, each of the exposed portions 256e is disposed with the coated portion 256f interposed therebetween. Although not shown, the first wiring 35a is connected to one exposed portion 255e by soldering, and the second wiring 35b is connected to one exposed portion 256e by soldering.

According to this embodiment, the substrate 250 includes a conductive portion 254 having conductivity, and a coating layer 257 that covers a part of the conductive portion 254, in the winding portion 252, the conductive portion 254 has the plurality of exposed portions 255e and 256e exposed radially outward, and coated portions 255f and 256f covered with the coating layer 257, and in the circumferential direction, the plurality of exposed portions 255e and 256e are respectively disposed with the coated portions 255f and 256f interposed therebetween. Therefore, since the plurality of exposed portions 255e and 256e are disposed in the circumferential direction, the degree of freedom in the position at which the wiring portion 35 is connected to the conductive portion 254 can be increased in the circumferential direction. Therefore, in the fourth step S4 of the assembly process of the endoscopic imaging device 210, since the wiring portion 35 can be easily connected to the conductive portion 254, it is possible to curb an increase in the number of assembly steps for the endoscopic imaging device 210.

According to this embodiment, when seen in the radial direction, each of the plurality of exposed portions 255e and 256e has a rectangular shape and is disposed at intervals in the circumferential direction. Therefore, when seen in the radial direction, since the exposed portions 255e and 256e are surrounded by the coating layer 257, when the wiring portion 35 and the exposed portions 255e and 256e are connected by soldering, it is possible to curb a flow of molten solder in the circumferential direction and the optical axis direction by the coating layer 257. Therefore, since the wiring portion 35 and the conductive portion 254 can be more easily connected by soldering, it is possible to more preferably curb an increase in the number of manufacturing steps for the endoscopic imaging device 210.

Further, according to this embodiment, as described above, a portion of the first coating layer 258 in which the hole 258a is not provided in the circumferential direction is fixed to the first curved portion 55b from an end portion of the first curved portion 55b on the distal end side to an end portion thereof on the proximal end side. Further, a portion of the second coating layer 259 in which the hole 259a is not provided in the circumferential direction is fixed to the second curved portion 56b from an end portion of the second curved portion 56b on the distal end side to an end portion thereof on the proximal end side. Therefore, the conductive portion 254 can be more firmly fixed to the base layer 53 by the coating layer 257. Therefore, in the third step S3 of the assembly process of the endoscopic imaging device 210, when the winding portion 252 is fixed to the holding frame 12, peeling of the conductive portion 254 from the base layer 53 can be more preferably curbed. Therefore, since the wiring portion 35 and the conductive portion 254 can be easily connected by soldering, it is possible to more preferably curb an increase in the number of assembly steps for the endoscopic imaging device 210.

### Second Modified Example

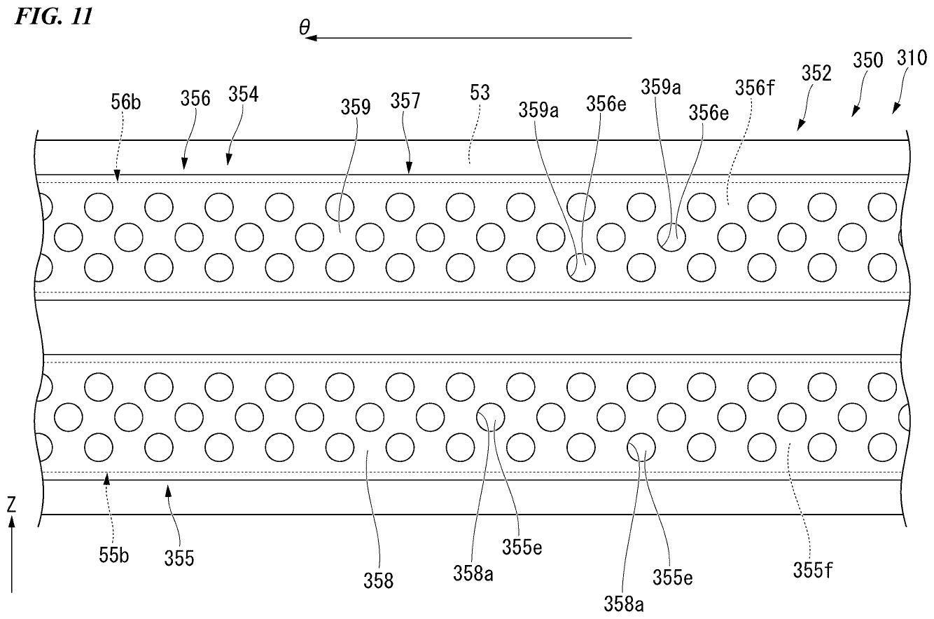

FIG. 11 is a side view showing a part of a winding portion 352 of an endoscopic imaging device 310 according to a second modified example of the first embodiment. In the following description, the same reference numerals are given to the same components as in the first embodiment described above, and the description thereof will be omitted.

As shown in FIG. 11, in this modified example, a substrate 350 includes a base layer 53, a conductive portion 354, and a coating layer 357. Configurations and the like of the base layer 53 in this modified example are the same as those of the base layer 53 in the first embodiment described above.

The conductive portion 354 is formed on the base layer 53. The conductive portion 354 includes a first conductive portion 355 and a second conductive portion 356. Other configurations of the conductive portion 354 are the same as those of the conductive portion 54 of the first embodiment described above.

The first conductive portion 355 includes a first extending portion 55a and a first curved portion 55b which are not shown. The second conductive portion 356 includes a second extending portion 56a and a second curved portion 56b which are not shown. Configurations and the like of the first extending portion 55a, the first curved portion 55b, the second extending portion 56a, and the second curved portion 56b are the same as those of the first extending portion 55a, the first curved portion 55b, the second extending portion 56a, and the second curved portion 56b of the first embodiment described above.

The coating layer 357 is formed across the base layer 53 and the conductive portion 354. The coating layer 357 covers a part of the conductive portion 354 from the outside in the radial direction. In the winding portion 352, the coating layer 357 includes a first coating layer 358 that covers a part of the first curved portion 55b from the outside in the radial direction, and a second coating layer 359 that covers a part of the second curved portion 56b from the outside in the radial direction.

A hole 358a that passes through the first coating layer 358 in the radial direction is provided in the first coating layer 358. The hole 358a has a substantially circular shape when seen in the radial direction. The shape of the hole 358a may be other shapes such as a rectangular shape. A plurality of holes 358a are provided in the first coating layer 358. Each of the holes 358a is disposed in a staggered manner at intervals from each other in the circumferential direction. When seen in the radial direction, each of the holes 358a is disposed to overlap the first curved portion 55b. Other configurations and the like of the first coating layer 358 are the same as those of the first coating layer 258 of the first modified example of the first embodiment described above.

A hole 359a that passes the second coating layer 359 in the radial direction is provided in the second coating layer 359. The hole 359a has a substantially circular shape when seen in the radial direction. The shape of the hole 359a may be other shapes such as a rectangular shape. A plurality of holes 359a are provided in the second coating layer 359. Each of the holes 359a is disposed in a staggered manner at intervals from each other in the circumferential direction. When seen in the radial direction, each of the holes 359a is disposed to overlap the second curved portion 56b. Other configurations and the like of the second coating layer 359 are the same as those of the second coating layer 259 of the first modified example of the first embodiment described above.

The first curved portion 55b has a plurality of exposed portions 355e exposed to the outside in the radial direction, and a coated portion 355f covered with the first coating layer 358 from the outside in the radial direction. The second curved portion 56b includes a plurality of exposed portions 356e and a coated portion 356f. That is, in the winding portion 352, the conductive portion 354 includes the plurality of exposed portions 355e and 356e and coated portions 355f and 356f.

Each of the plurality of exposed portions 355e is a portion of the first curved portion 55b that overlaps the plurality of holes 358a in the radial direction. Each of the plurality of exposed portions 356e is a portion of the second curved portion 56b that overlaps the plurality of holes 359a in the radial direction. When seen in the radial direction, each of the exposed portions 355e and 356e has a substantially circular shape. The exposed portions 355e are disposed in a staggered manner at intervals in the circumferential direction. In the circumferential direction, each of the exposed portions 355e is disposed with the coated portion 355f interposed therebetween. The exposed portions 356e are disposed in a staggered manner at intervals from each other in the circumferential direction. In the circumferential direction, each of the exposed portions 356e is disposed with the coated portion 356f interposed therebetween. Although not shown, the first wiring 35a is connected to one exposed portion 355e by soldering, and the second wiring 35b is connected to one exposed portion 356e by soldering.

According to this embodiment, the plurality of exposed portions 355e and 356e are disposed in a staggered manner in the circumferential direction. Therefore, since the plurality of exposed portions 355e and 356e are disposed in the circumferential direction, the degree of freedom in the position at which the wiring portion 35 is connected to the conductive portion 354 can be increased in the circumferential direction. Therefore, in the fourth step S4 of the assembly process of the endoscopic imaging device 310, since the wiring portion 35 can be easily connected to the conductive portion 354, it is possible to curb an increase in the number of assembly steps for the endoscopic imaging device 310.

Furthermore, in this embodiment, when seen in the radial direction, since the exposed portions 355e and 356e are surrounded by the coating layer 357, when the wiring portion 35 and the exposed portions 355e and 356e are connected by soldering, it is possible to curb the flow of the melted solder in the circumferential direction and the optical axis direction by the coating layer 357. Therefore, since the wiring portion 35 and the conductive portion 354 can be more easily connected by soldering, it is possible to more preferably curb an increase in the number of manufacturing steps for the endoscopic imaging device 310.

### Third Modified Example

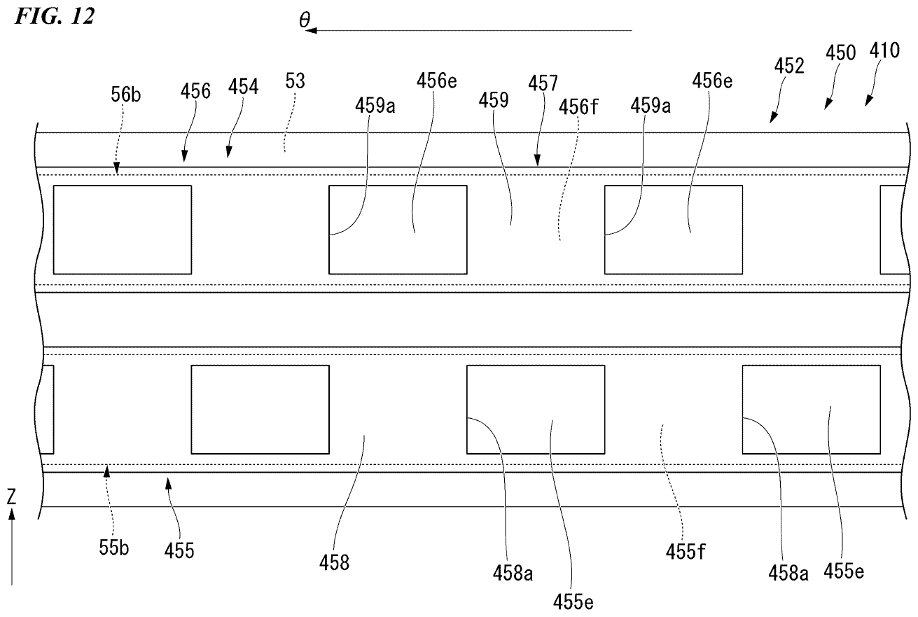

FIG. 12 is a side view showing a part of a winding portion 452 of an endoscopic imaging device 410 according to a third modified example of the first embodiment. In the following description, the same reference numerals are given to the same components as in the first embodiment described above, and the description thereof will be omitted.

As shown in FIG. 12, in this modified example, a substrate 450 includes a base layer 53, a conductive portion 454, and a coating layer 457. Configurations and the like of the base layer 53 in this modified example are the same as those of the base layer 53 in the first embodiment described above.

The conductive portion 454 is formed on the base layer 53. The conductive portion 454 includes a first conductive portion 455 and a second conductive portion 456. Other configurations of the conductive portion 454 are the same as those of the conductive portion 54 of the first embodiment described above.

The first conductive portion 455 includes a first extending portion 55a and a first curved portion 55b which are not shown. The second conductive portion 456 includes a second extending portion 56a and a second curved portion 56b which are not shown. The first curved portion 55b and the second curved portion 56b are disposed adjacent to each other in the optical axis direction. That is, the winding portion 452 includes a plurality of conductive portions 455 and 456 that are disposed adjacent to each other in the optical axis direction. Configurations of the first extending portion 55a, the first curved portion 55b, the second extending portion 56a, and the second curved portion 56b are the same as those of the first extending portion 55a, the first curved portion 55b, the second extending portion 56a, and the second curved portion 56b of the first embodiment described above.

The coating layer 457 is formed across the base layer 53 and the conductive portion 454. The coating layer 457 covers a part of the conductive portion 454 from the outside in the radial direction. In the winding portion 452, the coating layer 457 includes a first coating layer 458 that covers a part of the first curved portion 55b from the outside in the radial direction, and a second coating layer 459 that covers a part of the second curved portion 56b from the outside in the radial direction.

A hole 458a that passes through the first coating layer 458 in the radial direction is provided in the first coating layer 458. When seen in the radial direction, the hole 458a has a rectangular shape with long sides extending in the circumferential direction. The shape of the hole 458a may be other shapes such as a circular shape. A plurality of holes 458a are provided in the first coating layer 458. The holes 458a are disposed at intervals in the circumferential direction. When seen in the radial direction, each of the holes 458a is disposed to overlap the first curved portion 55b. Other configurations and the like of the first coating layer 458 are the same as those of the first coating layer 258 of the first modified example of the first embodiment described above.

A hole 459a that passes through the second coating layer 459 in the radial direction is provided in the second coating layer 459. When seen in the radial direction, the hole 459a has a rectangular shape with long sides extending in the circumferential direction. The shape of the hole 459a may be other shapes such as a circular shape. A plurality of holes 459a are provided in the second coating layer 459. The holes 459a are disposed at intervals in the circumferential direction. When seen in the radial direction, each of the holes 459a is disposed to overlap the second curved portion 56b. When seen in the optical axis direction, each of the holes 458a and each of the holes 459a do not overlap at least partially. In this embodiment, each of the holes 459a and each of the holes 458a are disposed at positions that do not overlap each other when seen in the optical axis direction. When seen in the optical axis direction, a part of each of the holes 459a may overlap a part of each of the holes 458a. Other configurations and the like of the second coating layer 459 are the same as those of the second coating layer 259 of the first modified example of the first embodiment described above.

The first curved portion 55b includes a plurality of exposed portions 455e exposed to the outside in the radial direction, and a coated portion 455f covered with the first coating layer 458 from the outside in the radial direction. The second curved portion 56b includes a plurality of exposed portions 456e and a coated portion 456f. That is, in the winding portion 452, the conductive portion 454 includes a plurality of exposed portions 455e and 456e and coated portions 455f and 456f.

Each of the plurality of exposed portions 455e is a portion of the first curved portion 55b that overlaps the plurality of holes 458a in the radial direction. Each of the plurality of exposed portions 456e is a portion of the second curved portion 56b that overlaps the plurality of holes 459a in the radial direction. When seen in the radial direction, each of the exposed portions 455e and 456e has a rectangular shape with long sides extending in the circumferential direction. The exposed portions 455e are disposed at intervals from each other in the circumferential direction. In the circumferential direction, each of the exposed portions 455e is disposed with the coated portion 455f interposed therebetween. The exposed portions 456e are disposed at intervals from each other in the circumferential direction. In the circumferential direction, each of the exposed portions 456e is disposed with the coated portion 456f interposed therebetween. When seen in the optical axis direction, each of the exposed portions 455e and each of the exposed portions 456e do not overlap at least partially. In this embodiment, each of the exposed portions 456e is disposed at a position that does not overlap each of the exposed portions 455e when seen in the optical axis direction. When seen in the optical axis direction, a part of each of the exposed portions 456e may overlap a part of each of the exposed portions 455e. Although not shown, the first wiring 35a is connected to one exposed portion 455e by soldering, and the second wiring 35b is connected to one exposed portion 456e by soldering.

According to this embodiment, the winding portion 452 has a plurality of conductive portions 455 and 456 disposed adjacent to each other in the optical axis direction, and when seen in the optical axis direction, the exposed portions 455e and 456e of the plurality of conductive portions 455 and 456 do not overlap at least partially. In other words, since at least a part of the exposed portion 455e and at least a part of the exposed portion 456e are disposed to be shifted in the circumferential direction, in the circumferential direction, a position at which the first wiring 35a and the first conductive portion 455 are connected can be shifted from a position at which the second wiring 35b and the second conductive portion 456 are connected. Therefore, in the fourth step S4 of the assembly process of the endoscopic imaging device 410, when the first wiring 35a and the first conductive portion 455 are connected by soldering, and when the second wiring 35b and the second conductive portion 456 are connected by soldering, it is possible to curb interference between the first wiring 35a and the second wiring 35b. Therefore, since the wiring portion 35 can be more easily connected to the conductive portion 454, it is possible to more preferably curb an increase in the number of assembly steps for the endoscopic imaging device 410.

Moreover, in this embodiment, since the plurality of exposed portions 455e and 456e are disposed in the circumferential direction, the degree of freedom in the position at which the wiring portion 35 is connected to the conductive portion 454 can be increased in the circumferential direction. Therefore, in the fourth step S4 of the assembly process of the endoscopic imaging device 410, since the wiring portion 35 can be easily connected to the conductive portion 454, it is possible to curb an increase in the number of assembly steps for the endoscopic imaging device 410.

Furthermore, in this embodiment, when seen in the radial direction, since the exposed portions 455e and 456e are surrounded by the coating layer 457, when the wiring portion 35 and the exposed portions 455e and 456e are connected by soldering, the flow of the melted solder in the circumferential direction and the optical axis direction can be curbed by the coating layer 457. Therefore, since the wiring portion 35 and the conductive portion 454 can be more easily connected by soldering, it is possible to more preferably curb an increase in the number of manufacturing steps for the endoscopic imaging device 410.

Although the embodiments of the present invention have been described above, the configurations and combinations thereof in the embodiments are merely examples, and additions, omissions, substitutions, and other changes to the configurations are possible without departing from the spirit of the present invention. Moreover, the present invention is not limited by the embodiments.

The configuration of the substrate is not limited to this embodiment. For example, the substrate does not need to have the straight portion. In this case, the cable is connected to the winding portion. Further, the number of conductive portions included in the substrate is not limited to two, and may be one, or three or more.

The method for manufacturing an endoscopic imaging device is not limited to this embodiment. For example, the work of fixing the straight portion of the substrate to the lens barrel part in the third step may be performed before the first step. In this case, before the first step, after the winding portion of the substrate is formed into a curved shape in the circumferential direction, and the straight portion of the substrate is fixed to the lens barrel part, in the first step, the holding frame is rotated in the circumferential direction with respect to the lens barrel part to adjust the position of the optical system. Furthermore, after the second step, the winding portion of the substrate is fixed to the holding frame.

What is claimed is:

1. An endoscopic imaging device comprising:
an optical system;
a tubular holding frame configured to accommodate a part of the optical system therein and to extend in an optical axis direction;
an electronic component having a wiring portion and held in the holding frame; and
a substrate having a winding portion configured to extend in a circumferential direction along an outer circumferential surface of the holding frame, and a straight portion configured to extend from the winding portion to one side in the optical axis direction,
wherein the substrate has a conductive portion disposed across the winding portion and the straight portion, and
the wiring portion is connected to the conductive portion disposed in the winding portion.

2. The endoscopic imaging device according to claim 1, wherein the wiring portion is connected to a portion of the conductive portion near a position at which the wiring portion is pulled out of the electronic component.

3. The endoscopic imaging device according to claim 1, wherein the electronic component includes a movable frame disposed inside the holding frame and configured to hold a part of the optical system therein, a yoke provided outside the holding frame, a pair of magnets disposed on one side of the yoke in the optical axis direction and on the other side thereof in the optical axis direction so that the same magnetic poles face each other in the optical axis direction, and a coil main body portion accommodated inside the yoke, and
the movable frame is made of a magnetic body and is movable in the optical axis direction.

4. The endoscopic imaging device according to claim 3, wherein a notch portion is provided in the yoke, and
the wiring portion is pulled out from the notch portion to an outside of the yoke.

5. The endoscopic imaging device according to claim 1, comprising a tubular lens barrel part configured to extend in the optical axis direction,
wherein the lens barrel part holds the holding frame, and
a part of the straight portion is fixed to an outer circumferential surface of the lens barrel part, and the winding portion is fixed to the holding frame.

6. The endoscopic imaging device according to claim 1, wherein the substrate has a conductive portion having conductivity and a coating layer which covers a part of the conductive portion,
in the winding portion, the conductive portion has an exposed portion exposed to an outside in a radial direction and a coated portion covered with the coating layer, and
the exposed portion extends in the circumferential direction.

7. The endoscopic imaging device according to claim 1, wherein the substrate has a conductive portion having conductivity and a coating layer covering a part of the conductive portion,
in the winding portion, the conductive portion has a plurality of exposed portions exposed to the outside in a radial direction and a coated portion covered with the coating layer, and
in the circumferential direction, each of the plurality of exposed portions is disposed with the coated portion interposed therebetween.

8. The endoscopic imaging device according to claim 7, wherein, when seen in a radial direction, the respective plurality of exposed portions have a rectangular shape and are disposed at a uniform interval in the circumferential direction.

9. The endoscopic imaging device according to claim 7, wherein the plurality of exposed portions are disposed in a staggered manner in the circumferential direction.

10. The endoscopic imaging device according to claim 7, wherein the winding portion has a plurality of the conductive portions disposed adjacent to each other in the optical axis direction, and
the exposed portions of the plurality of conductive portions do not overlap each other at least partially when seen in the optical axis direction.

11. The endoscopic imaging device according to claim 3, wherein the wiring portion has a first wiring and a second wiring,
a length of the first wiring is longer than a length of the second wiring,
the electronic component is disposed closer to the other side than the winding portion in the optical axis direction,
the conductive portion includes a first conductive portion and a second conductive portion,
in the winding portion, each of the first conductive portion and the second conductive portion extends in the circumferential direction, and the first conductive portion is disposed closer to one side than the second conductive portion in the optical axis direction, and
the first wiring is connected to the first conductive portion, and the second wiring is connected to the second conductive portion.

12. An endoscope comprising:
an inserting part inserted into a subject; and
an endoscopic imaging device provided at a distal end of the inserting part,
wherein the endoscopic imaging device includes an optical system which forms an optical image, an electronic component having a wiring portion, a tubular holding frame which accommodates a part of the optical system therein and extends in an optical axis direction, and a substrate having a winding portion which extends in a circumferential direction along an outer circumferential surface of the holding frame, and a straight portion which extends from the winding portion to one side in the optical axis direction, and
the wiring portion is connected to a portion of the winding portion near a position at which the wiring portion is pulled out of the electronic component.

13. A method for manufacturing an endoscopic imaging device which includes an optical system that forms an optical image, a tubular holding frame that accommodates a part of the optical system therein and extends in an optical axis direction, an electronic component having a wiring portion and held in the holding frame, a lens barrel part that holds the holding frame, and a substrate having a winding portion that extends in a circumferential direction along an outer circumferential surface of the holding frame, and a straight portion that extends from the winding portion to one side in the optical axis direction, wherein the wiring portion is connected to a portion of the winding portion near a position at which the wiring portion is pulled out of the electronic component, the method comprising:
a first step of adjusting a position of the optical system by rotating the holding frame in the circumferential direction with respect to the lens barrel part;
a second step of fixing the holding frame to the lens barrel part;
a third step of fixing the straight portion to the lens barrel part and fixing the winding portion to the holding frame; and
a fourth step of connecting the wiring portion to the winding portion.
